(19) United States
(12) Patent Application Publication
(10) Pub. No.: US 2026/0166753 A1
(43) Pub. Date: Jun. 18, 2026

(54) TROCAR HOLDER FOR SURGICAL ROBOT

(71) Applicant: LIVSMED INC., Seongnam-si (KR)

(72) Inventors: Dong Hyuk KIM, Seoul (KR); Jae Hoon SHIN, Gwangju-si (KR); Yeon Taek JEONG, Suwon-si (KR); Young Wan KIM, Seongnam-si (KR)

(21) Appl. No.: 19/415,222

(22) Filed: Dec. 10, 2025

(30) Foreign Application Priority Data
Dec. 12, 2024 (KR) 10-2024-0184352

(51) Int. Cl. B25J 15/00; A61B 17/34; A61B 34/30

(52) U.S. Cl. CPC B25J 15/0033; A61B 17/34; A61B 34/30

(57) ABSTRACT

A trocar holder for a surgical robot includes a clamp assembly including a first clamp and a second clamp which are arranged to face each other and configured to be rotatable around different shafts, a sliding member connected to the first clamp and the second clamp, and configured to move between a proximal end and a distal end along a first axis to cause rotational motions of the first clamp and the second clamp, and a holder body in which the sliding member and at least a portion of the clamp assembly are disposed.

### CROSS-REFERENCE TO RELATED APPLICATION

This application is based on and claims priority under 35 U.S.C. § 119 to Korean Patent Application No. 10-2024-0184352, filed on Dec. 12, 2024, in the Korean Intellectual Property Office, the disclosure of which is incorporated by reference herein in its entirety.

### BACKGROUND

### 1. Field

The present disclosure relates to a trocar holder for a surgical robot.

### 2. Description of the Related Art

In medical terms, surgery refers to curing an illness by cutting, incising, or manipulating the skin, mucous membranes, or other tissues by using medical devices. In particular, open surgery, which involves cutting open the skin at the surgical site and treating, shaping, or removing the organs inside, may cause problems such as bleeding, side effects, patient pain, and scarring. Therefore, surgery using a robot or surgery performed by forming a small hole in the skin and inserting only a medical device, such as a laparoscope, a surgical instrument, or a microsurgical microscope, has recently attracted attention as an alternative.

Surgical robots refer to robots which have the function of replacing surgical operations performed by surgeons. Such surgical robots have the advantages of being able to perform more accurate and precise motions than humans and perform remote surgeries.

Surgical robots which have been currently developed worldwide include bone surgical robots, laparoscopic surgical robots, stereotactic surgical robots, and the like. Laparoscopic surgical robots are robots which perform minimally invasive surgery using a laparoscope and small surgical tools.

Surgical robots usually include a master robot and a slave robot. When the surgeon manipulates the control lever (e.g., handle) equipped on the master robot, the surgical tool coupled to the robot arm of the slave robot or held by the robot arm is manipulated to perform the surgery.

Laparoscopic surgery is a cutting-edge surgical technique which drills a small hole in a navel area, inserts a laparoscope, which is an endoscope for looking inside the abdomen, into the small hole, and performs surgery. Laparoscopic surgery is a field which is expected to see great development in the future. Recent laparoscopes are capable of obtaining clearer and more magnified images than visual inspection by using computer chips mounted thereon. Furthermore, advances have made it possible to perform any type of surgery by using specially designed laparoscopic surgical instruments while viewing a screen through a monitor.

Laparoscopic surgery is performed by making a tube leading to a patient's abdomen using a surgical instrument called a trocar and then inserting a laparoscope, surgical instruments, or the like into an abdominal surgical site through the trocar, allowing observation of the abdominal surgical site.

The aforementioned background technology is technical information possessed by the inventor for derivation of the present disclosure or acquired by the inventor during the derivation of the present disclosure, and is not necessarily prior art disclosed to the public before the application of the present disclosure.

### SUMMARY

The present disclosure provides a trocar holder applicable to a surgical robot for use in laparoscopic surgery or various other surgeries, which facilitates the mounting and unmounting of a trocar and improves the fastening force of the trocar.

According to an embodiment, a trocar holder for a surgical robot may include a clamp assembly including a first clamp and a second clamp which are arranged to face each other and configured to be rotatable around different shafts, a sliding member connected to the first clamp and the second clamp and configured to move between a proximal end and a distal end along a first axis to cause rotational motions of the first clamp and the second clamp, and the holder body in which the sliding member and at least a portion of the clamp assembly are disposed.

In another embodiment, the clamp assembly may include reaction members respectively disposed on the first clamp and the second clamp and configured to contact the sliding member, and the reaction members and the sliding member may be configured to move relative to each other by a force acting between the reaction members and the sliding member.

In the other embodiment, the sliding member may include a pair of first inclined portions formed symmetrically with respect to the first axis at a front end, and the reaction members may come into contact with a respective one of the pair of first inclined portions and may be pressed by the respective one of the pair of first inclined portions or may press the respective one of the pair of first inclined portions.

In the other embodiment, when the sliding members move forward in a direction toward the distal end, the pair of first inclined portions may press the reaction members, and when the reaction members move closer to the first axis, the reaction members may press the pair of first inclined portions.

In the other embodiment, when the sliding member moves forward in the direction toward the distal end, the reaction members may move in a direction away from the first axis, and the first clamp and the second clamp may be internally rotated.

In the other embodiment, when the first clamp and the second clamp are externally rotated, the reaction members may move in a direction closer to the first axis, and the sliding member may move backward in a direction toward the proximal end.

In the other embodiment, the sliding member may include a pair of guide holes having at least one surface including the respective one of the pair of first inclined portions, and at least one portion of each of the reaction members may be inserted into the respective one of the pair of guide holes and may be movable therein.

In the other embodiment, a distance between the pair of first inclined portions may increase from a distal end side toward a proximal end side.

In the other embodiment, the reaction members may have a cylindrical shape, and a side surface of each of the reaction members may contact the sliding member.

In the other embodiment, the first clamp may include a first grip portion extending from a rotation center of the first clamp and a first acting portion extending to an opposite side of the first grip portion with respect to the rotation center of the first clamp, and the second clamp may include a second grip portion extending from a rotation center of the second clamp and a second acting portion extending to an opposite side of the second grip portion with respect to the rotation center of the second clamp.

In the other embodiment, the reaction members may respectively be disposed on each of the first acting portion and the second acting portion.

In the other embodiment, each of the first acting portion and the second acting portion may provide a holding region configure to be pressed from an outside, and when the holding region is pressed, the first grip portion and the second grip portion may be rotated in a direction away from each other.

In the other embodiment, the first clamp and the second clamp may be rotated by the sliding member while maintaining a symmetrical state.

In the other embodiment, the holder body may include a first elastic member configured to press the sliding member in a direction toward the distal end.

In the other embodiment, the holder body may further include a pusher member configured to move along a second axis parallel to the first axis, and a second elastic member configured to press the pusher member in the direction toward the distal end, and the pusher member may be configured to contact a mounting portion of a trocar when the trocar is mounted on the trocar holder.

In the other embodiment, the pusher member may press the mounting portion in the direction toward the distal end.

In the other embodiment, each of the first clamp and the second clamp may include a fastening portion formed to be curved inwardly at a side toward the distal end, and the fastening portion may be configured to contact the trocar and restrict movement of the trocar toward the distal end.

In the other embodiment, the holder body may further include a first detector configured to detect whether the trocar is mounted, and the first detector may be operated when the pusher member is moved backward in a direction toward the proximal end.

In the other embodiment, a second inclined portion bent toward a space between the first clamp and the second clamp may be formed at an end portion of each of the first clamp and the second clamp toward the distal end.

In the other embodiment, each of the first clamp and the second clamp may include an inner surface corresponding to a shape of a mounting portion of the trocar.

Other aspects, features, and advantages of the present disclosure will become better understood through the accompanying drawings, the appended claims, and the detailed description.

### BRIEF DESCRIPTION OF THE DRAWINGS

The above and other aspects, features, and advantages of certain embodiments of the present disclosure will be more apparent from the following description taken in conjunction with the accompanying drawings.

FIG. 1 is a conceptual diagram illustrating a surgical robot system including a surgical robot, according to an embodiment.

FIG. 2 is a perspective view of the surgical robot of FIG. 1.

FIG. 3 is an enlarged perspective view illustrating a partial configuration of a first arm assembly of FIG. 2.

FIG. 4 is a perspective view illustrating a trocar holder and a trocar, according to an embodiment.

FIG. 5 is a perspective view illustrating a state in which the trocar is mounted on the trocar holder in FIG. 4.

FIG. 6 is a perspective view illustrating a mounting portion of the trocar of FIG. 4.

FIG. 7 is a perspective view illustrating a trocar holder according to an embodiment.

FIG. 8 is a perspective view illustrating a state in which an upper plate is removed from the trocar holder of FIG. 7.

FIG. 9 is an exploded perspective view illustrating a sliding member separated from the trocar holder of FIG. 8.

FIG. 10 is a cross-sectional view illustrating a cross-section of the trocar holder of FIG. 8.

FIG. 11 is a diagram illustrating a force acting between a sliding member and a clamp of a trocar holder according to an embodiment.

FIG. 12 is a cross-sectional view illustrating an opening operation of the trocar holder of FIG. 10.

FIG. 13 is a perspective view illustrating the trocar holder and the mounting portion of the trocar of FIG. 5.

FIG. 14 is an exploded perspective view illustrating a partial configuration including a pusher member of the trocar holder of FIG. 13.

FIG. 15 is an enlarged perspective view illustrating a clamp of the trocar holder of FIG. 13.

FIGS. 16 to 18 are diagrams illustrating a state of a trocar holder when a trocar is mounted on a trocar holder, according to an embodiment.

### DETAILED DESCRIPTION

Hereinafter, the following embodiments will be described in detail with reference to the accompanying drawings. When describing the embodiments with reference to the accompanying drawings, the same or corresponding elements are denoted by the same reference numerals and redundant descriptions thereof are omitted.

Because various changes may be made to the present embodiments, specific embodiments will be illustrated in the drawings and described in detail in the detailed description. Effects and features of the present embodiments, and methods of achieving them will be clarified with reference to the detailed description below along with the drawings. However, the present embodiments are not limited to the embodiments disclosed below and may be implemented in various forms.

In describing the present disclosure, when the detailed description of the relevant known technology is determined to obscure the gist of the present disclosure, the detailed description thereof may be omitted.

The singular forms as used herein are intended to include the plural forms as well unless the context clearly indicates otherwise. While the terms such as “first” and “second” may be used to describe various elements, the elements should not be limited by the terms. These terms are only used to distinguish one element from another.

In the following embodiments, it will be understood that the terms “include” and/or “comprise” used herein specify the presence of stated features or elements, but do not preclude the presence or addition of one or more other features or elements.

In the following embodiments, it will be understood that, when a portion such as unit, region, or element is referred to as being “on” another portion, this may include not only a case where the portion is directly on the other portion, but also a case where intervening units, regions, or elements may be present therebetween.

In the following embodiments, it will be understood that the terms “connection” or “coupling” do not necessarily mean “direct and/or fixed connection or coupling” of two members, unless the context clearly indicates otherwise, and this does not preclude the arrangement of other members between the two members.

Also, sizes of elements in the drawings may be exaggerated or reduced for convenience of explanation. For example, because sizes and thicknesses of elements in the drawings are arbitrarily illustrated for convenience of explanation, the following embodiments are not limited thereto.

First, a surgical robot 10 capable of adopting a trocar holder according to an embodiment is described.

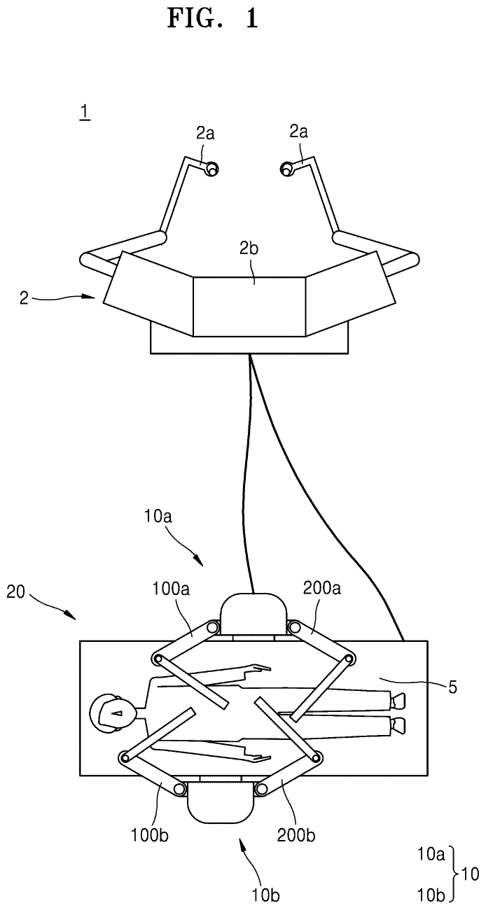

FIG. 1 is a conceptual diagram illustrating a surgical robot system 1 including a surgical robot, according to an embodiment.

Referring to FIG. 1, the surgical robot system 1 may include a master robot 2 and a surgical robot 10.

The master robot 2 may include a manipulation member and a display member, and the surgical robot 10 may include one or more robot arm assemblies 100 and 200.

The master robot 2 may include manipulation members 2a which allows an operating surgeon to hold and manipulate the master robot 2 with both hands. A display member 2b of the master robot 2 may display an image captured using a laparoscope as a video image. In some embodiments, a virtual manipulation panel may be displayed on the display member 2b together with the image captured using the laparoscope or the like, or may be displayed independently. A detailed description of the layout, configuration, or the like of the virtual manipulation panel is omitted.

In some embodiments, the surgical robot 10 may include at least two robot arm assemblies 100 and 200. The robot arm assemblies 100 and 200 may be provided in a module form which is operable independently of each other, and an algorithm for preventing collision between the robot arm assemblies 100 and 200 may be applied to the surgical robot system 1.

The surgical robot system 1 may include one or more surgical robots 10. FIG. 1 illustrates an embodiment in which the surgical robot system 1 includes two surgical robots 10a and 10b each having two robot arm assemblies 100 and 200, that is, a total of four robot arm assemblies 100a, 200a, 100b, and 200b are arranged.

In an embodiment, surgical instruments SI may be attached to two or more of the robot arm assemblies 100 and 200, and laparoscopes may be attached to one or more of the robot arm assemblies 100 and 200. An operating surgeon may select the robot arm assemblies 100a, 200a, 100b, and 200b to be controlled through the master robot 2. By allowing the operating surgeon to directly control three or more surgical instruments using the master robot 2, the operating surgeon may accurately and freely control multiple instruments on a surgical bed 5 according to the intention of the operating surgeon without the need for a surgical assistant.

A detailed configuration and operating principle of the surgical robot 10 are described in detail.

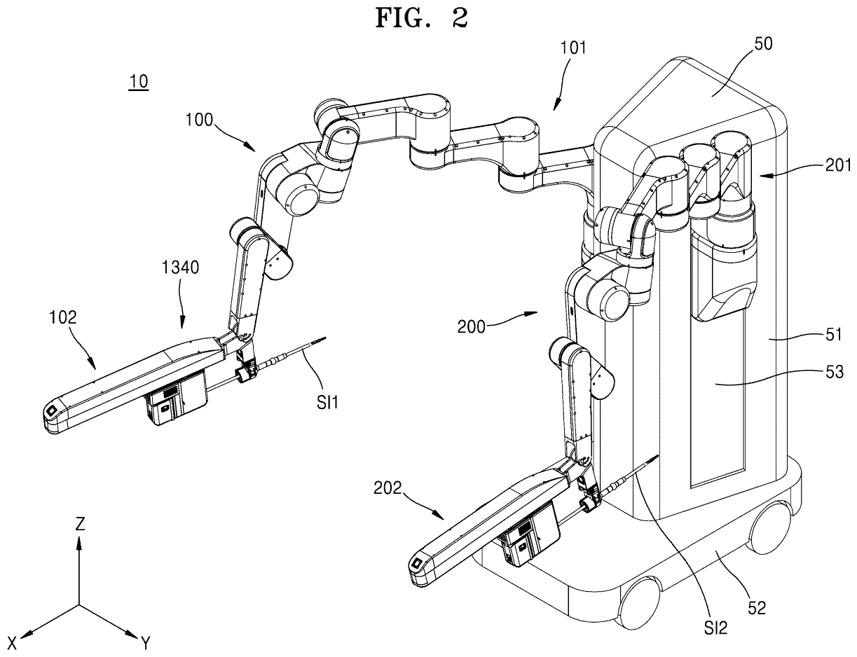

FIG. 2 is a perspective view of the surgical robot 10 of FIG. 1.

Referring to FIG. 2, the surgical robot 10 may include a main body part 50, a first arm assembly 100, and a second arm assembly 200. FIG. 2 illustrates an embodiment in which the surgical robot 10 includes two robot arm assemblies, and the respective robot arm assemblies are defined as the first arm assembly 100 and the second arm assembly 200.

The main body part 50 may act as the body of the surgical robot 10, and the first arm assembly 100 and the second arm assembly 200 may be arranged in the main body part 50. In some embodiments, the main body part 50 may provide reference points for driving the first arm assembly 100 and the second arm assembly 200.

The main body part 50 may include a first main body 51 and a second main body 52. The first arm assembly 100 and the second arm assembly 200 may be arranged in the first main body 51. The second main body 52 may support the first main body 51. In some embodiments, the second main body 52 may have wheels, as illustrated in FIG. 2. Due to this, the surgical robot 10 may be movable.

The main body part 50 may have an elevating guide 53. The elevating guide 53 may be provided to correspond to the number of robot arm assemblies arranged in the main body part 50. The elevating guide 53 may be formed concavely on a side of the main body part 50, and the respective robot arm assemblies 100 and 200 may be coupled to the elevating guide 53 and slidingly movable in a first direction.

In describing the present disclosure, a portion close to the main body part 50 is referred to as a proximal end, and a portion far from the main body part 50 is referred to as a distal end. For example, a portion of the first arm assembly 100 which is close to the main body part 50 is defined and described as a proximal end 101 of the first arm assembly 100, and a portion of the first arm assembly 100 which is far from the main body part 50 is defined and described as a distal end 102 of the first arm assembly 100. Similarly, a portion of the second arm assembly 200 which is close to the main body part 50 is defined and described as a proximal end 201 of the second arm assembly 200, and a portion of the second arm assembly 200 which is far from the main body part 50 is defined and described as a distal end 202 of the second arm assembly 200.

The first arm assembly 100 may be arranged on a side of the main body part 50, and a first surgical instrument SI1 may be mounted on the first arm assembly 100. The surgical robot 10 may drive the first arm assembly 100 to control the position and posture of the first surgical instrument SI1.

A first arm connection portion (not shown) may have a plurality of connection links, and the posture of the first arm connection portion may be determined according to the driving of the respective connection links. A remote center of motion (RCM) point of the first surgical instrument SI1 may be determined according to the posture of the first arm connection portion (not shown). In this case, the RCM point of the first surgical instrument SI1 may refer to a virtual center point which serves as a reference for the rotation of the first surgical instrument SI1. The first surgical instrument SI1 may be rotated around the RCM point to perform a yaw motion and a pitch motion.

The second arm assembly 200 may be arranged on another side of the main body part 50, and a second surgical instrument SI2 may be mounted on the second arm assembly 200. The surgical robot 10 may drive the second arm assembly 200 to control the position and posture of the second surgical instrument SI2.

A second arm connection portion (not shown) may have a plurality of connection links, and the posture of the second arm connection portion (not shown) may be determined according to the driving of the respective connection links. A RCM point of the second surgical instrument SI2 may be determined according to the posture of the second arm connection portion (not shown). In this case, the RCM point of the second surgical instrument SI2 may refer to a virtual center point which serves as a reference for the rotation of the second surgical instrument SI2. The second surgical instrument SI2 may be rotated around the RCM point to perform a yaw motion and a pitch motion.

The arm assemblies may each include a plurality of arm connection links and a plurality of arm extension links. The arm connection link and the arm extension link may be respectively rotated around reference axes thereof. Due to the rotation motion, the arm assemblies may control the posture and layout of the surgical instrument within a movable range.

A detailed description of the arm connection links and the arm extension links is omitted herein.

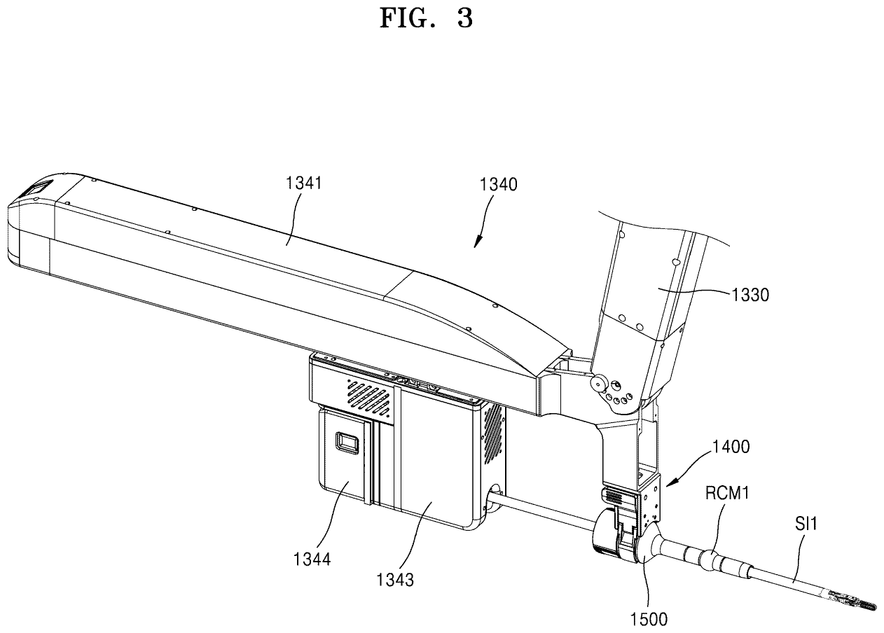

FIG. 3 is an enlarged perspective view illustrating a partial configuration of the first arm assembly 100 of FIG. 2.

Referring to FIGS. 2 and 3, the first arm assembly 100 according to an embodiment may include a first arm slide link 1340 on the distal end 102 side.

The first arm slide link 1340 may slidingly move the first surgical instrument SI1.

The first arm slide link 1340 may be coupled to another end of a first-arm third-extension link 1330, that is, on a distal end side, and the first surgical instrument SI1 may be arranged in the first arm slide link 1340.

The first arm slide link 1340 may include a translation arm 1341, a slide motor pack 1343, a slide driving unit 1344, and a trocar holder 1400.

The translation arm 1341 may be coupled to another end of the first-arm third-extension link 1330 and may be moved together with the first-arm third-extension link 1330. For example, when the first-arm third-extension link 1330 is driven, the posture of the translation arm 1341 may be changed together.

The slide motor pack 1343 may provide a driving force for the slide motion of the first surgical instrument SI1. The slide motor pack 1343 may include one or more first motors and other components capable of producing and transmitting power.

The slide driving unit 1344 may receive the driving force from the slide motor pack 1343 and slidingly move the first surgical instrument SI1. The first surgical instrument SI1 may have an end connected to the slide driving unit 1344 and may be linearly moved by the slide driving unit 1344.

The trocar holder 1400 may be arranged in an area of the translation arm 1341, and a trocar 1500 may be mounted on the trocar holder 1400. For example, the trocar holder 1400 may be arranged close to an end portion of the first-arm third-extension link 1330 on the distal end side.

The trocar 1500 may be mounted on the trocar holder 1400, and the first surgical instrument SI1 may be coupled to the trocar 1500. The first surgical instrument SI1 may be mounted by passing through the trocar 1500. In some embodiments, the first surgical instrument SI1 may be partially supported on the trocar 1500 and may be slidingly moved.

An RCM point RCM1 of the first surgical instrument SI1 may be formed on a side of the trocar 1500. For example, the trocar 1500 may provide an RCM point, which is a reference point for rotation, including a yaw motion and a pitch motion of the first surgical instrument SI1, on a side. When the posture of the first arm connection portion is determined, the position of the RCM point arranged on the trocar 1500 may also be determined, and even when the first surgical instrument SI1 is slidingly moved, the position of the RCM point may be fixed.

Hereinafter, a trocar holder and a trocar, according to an embodiment, are described in detail.

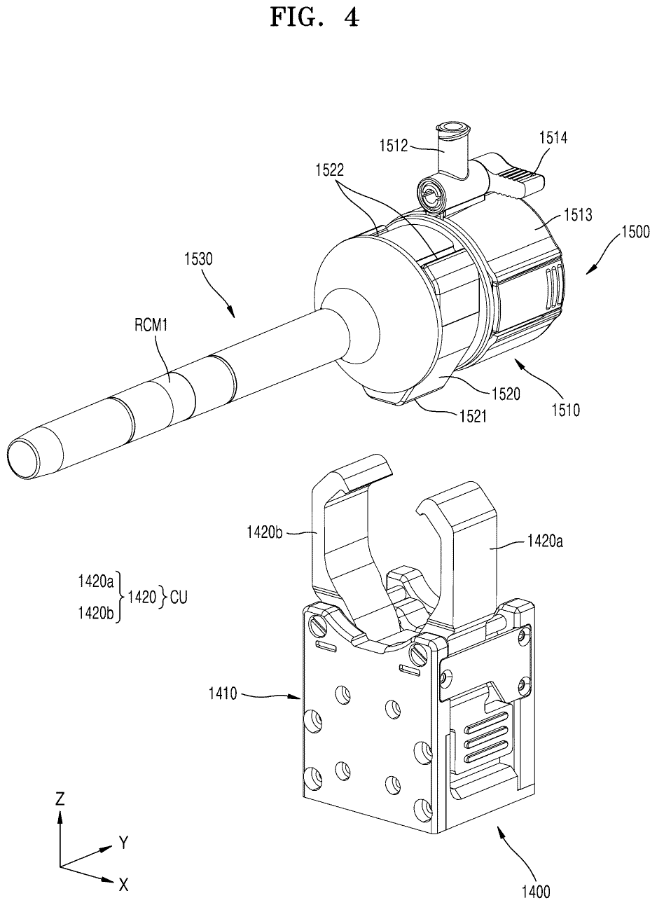

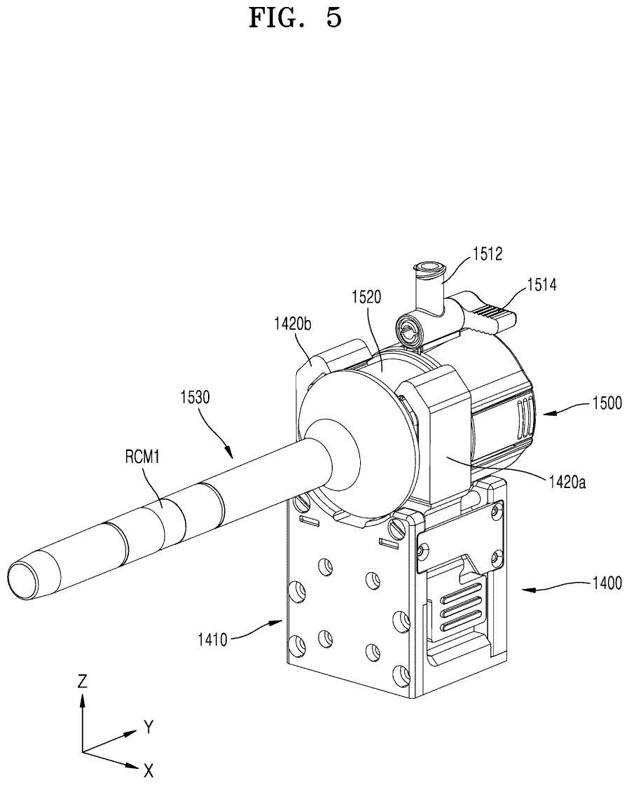

FIG. 4 is a perspective view illustrating a trocar holder and a trocar, according to an embodiment, and FIG. 5 is a perspective view illustrating a state in which the trocar is mounted on the trocar holder in FIG. 4.

First, a trocar 1500 according to an embodiment is described.

Referring to FIGS. 4 and 5, the trocar 1500 according to an embodiment may include a mounting portion 1520, an upper module 1510, and a cannula 1530.

The upper module 1510 is a structure with a hollow portion formed therein so that a surgical instrument may be inserted therethrough. In some embodiments, various components for the trocar 1500 to perform functions may be arranged inside the upper module 1510.

The upper module 1510 may include an inner case (not shown), a gas insertion tube 1512, and a cover part 1513.

The inner case may be formed in a hollow cylindrical shape and may accommodate components of the upper module 1510 therein. In some embodiments, the gas insertion tube 1512 to be described below may be connected to a side surface of the inner case.

The gas insertion tube 1512 may be formed to extend in one direction from the side surface of the inner case. Carbon dioxide may be supplied through the gas insertion tube 1512 so that carbon dioxide may be injected into the patient's body. The injected carbon dioxide may act to inflate the patient's abdominal cavity, which may ensure a space for laparoscopic surgery. The gas insertion tube 1512 may include a material such as plastic, or may include a soft, elastically deformable material so as to be elastically bent.

In some embodiments, a gas valve 1514 may be formed in the gas insertion tube 1512 to control opening or closing of the gas insertion tube 1512.

In some embodiments, the inner case may further include a check valve (not shown). The check valve may prevent gas (carbon dioxide) inserted into the patient's body through the gas insertion tube 1512 from leaking out of the trocar 1500. The check valve may be fixedly connected within the inner case and may be formed to be opened when the surgical instrument is inserted into the patient's body and to be closed when the surgical instrument is separated from the patient's body, which may prevent gas injected into the patient's body from leaking to the outside.

The inner case may be formed so that at least one area is coupled to the mounting portion 1520 to be described below. For example, an area of the inner case may be formed to be smaller than the inner diameter of the mounting portion 1520 so as to be inserted into the inside of the mounting portion 1520.

The cover part 1513 may be coupled to the upper portion of the inner case and may couple and seal the above-described members arranged inside the inner case. In an embodiment, the cover part 1513 may cover the side surface and the upper portion of the inner case.

The upper surface of the cover part 1513 may form an opening (not shown). For example, an opening through which the surgical instrument is inserted may be formed in the central portion of the cover part 1513. In other words, the opening of the cover part 1513 may act as an entrance of a communication tube which communicates the inner case with a hollow portion of the cannula 1530 to be described below.

In some embodiments, an end tool holding member (not shown) may be detachably attached to the upper portion of the cover part 1513. For example, a type of groove may be formed on the upper surface of the cover part 1513 so that additional components such as the end tool holding member is fastened thereto.

The cannula 1530 may be coupled to an end portion (e.g., a lower end portion) of the upper module 1510 and may have a hollow portion formed therein so that the surgical instrument may be inserted therethrough. The cannula 1530 may be inserted into the patient's body through the patient's navel or skin incision site. In this state, the surgical instrument passing through the upper module 1510 may pass through the cannula 1530 and enter the patient's body.

In some embodiments, the cannula 1530 may be provided with the RCM point RCM1 described above. For example, the cannula 1530 may provide the RCM point RCM1, which is a reference point for rotation, including a yaw motion and a pitch motion of the surgical instrument.

In some embodiments, the cannula 1530 may include the mounting portion 1520 to be described below. The mounting portion 1520, which is an area to be mounted on the trocar holder 1400, may be an area formed between the cannula 1530 and the upper module 1510.

For example, the mounting portion 1520 may be formed on the outer surface of the upper end portion of the cannula 1530. For example, the mounting portion 1520 may be formed integrally with the cannula 1530.

In another embodiment, the mounting portion 1520 may be a member which is detachable from the cannula 1530 and attachable to the cannula 1530. The mounting portion 1520, which is an area between the cannula 1530 and the upper module 1510, may connect the cannula 1530 to the upper module 1510. For example, the inner case described above may be inserted into the mounting portion 1520 so that the mounting portion 1520 and the inner case may be coupled to each other.

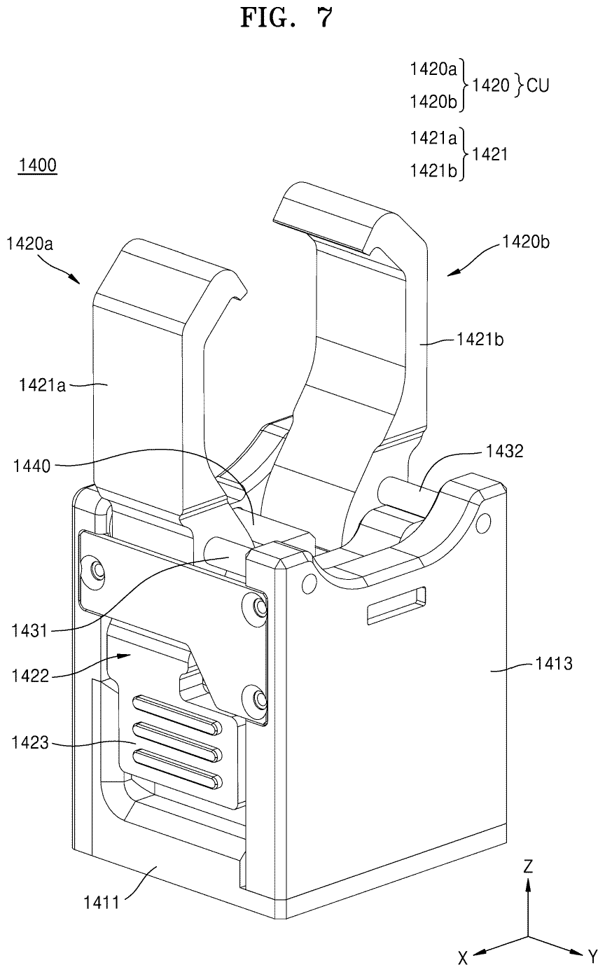

The mounting portion 1520 may be an area to be mounted on the trocar holder 1400. For example, the mounting portion 1520 may be a portion gripped by a clamp assembly 1420 of the trocar holder 1400. For example, the mounting portion 1520 may be arranged between a first grip portion (see 1421a of FIG. 7) of a first clamp 1420a and a second grip portion (see 1421b of FIG. 7) of a second clamp 1420b, which are to be described below, and may be fixed to the trocar holder 1400.

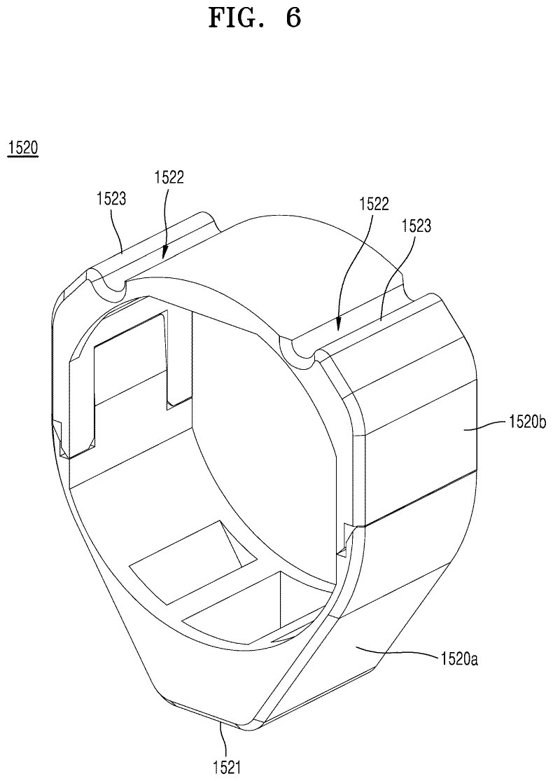

FIG. 6 is a perspective view illustrating the mounting portion 1520 of the trocar 1500 of FIG. 4.

Referring to FIG. 6, the mounting portion 1520 may be a member formed in a ring shape to surround an area of the trocar 1500, as described above. For example, the inner surface of the mounting portion 1520 may be formed in a shape corresponding to an area of the trocar 1500, for example, the outer surface of the cannula 1530.

The outer surface of the mounting portion 1520 may be formed in a shape entirely corresponding to the shapes of the inner surfaces of the first clamp 1420a and the second clamp 1420b. According to an embodiment, the side surface portion of the mounting portion 1520 which comes into contact with the inner surfaces of the first clamp 1420a and the second clamp 1420b may be provided in a shape matching the inner surfaces of the clamp assembly 1420.

In some embodiments, the mounting portion 1520 may include a pusher member contact part 1521 which comes into contact with the pusher member 1440 to be described below. The pusher member contact part 1521 is a part which comes into contact with the pusher member 1440 when the trocar 1500 is mounted on the trocar holder 1400 and presses the pusher member 1440. The pusher member contact part 1521 may form a contact surface opposite the pusher member 1440. For example, the pusher member contact part 1521 may be formed in a protruding shape, and thus, the overall shape of the mounting portion 1520 may have a shape protruding in the mounting direction of the trocar 1500. Therefore, a user may intuitively identify the mounting direction of the trocar 1500 through the shape of the mounting portion 1520.

In some embodiments, the mounting portion 1520 may include a groove 1522 formed in an opposite direction of the pusher member contact part 1521. In other words, the groove 1522 may be formed in a direction opposite to the mounting direction of the trocar 1500. For example, the groove 1522 may be symmetrically provided on a side surface of the mounting portion 1520.

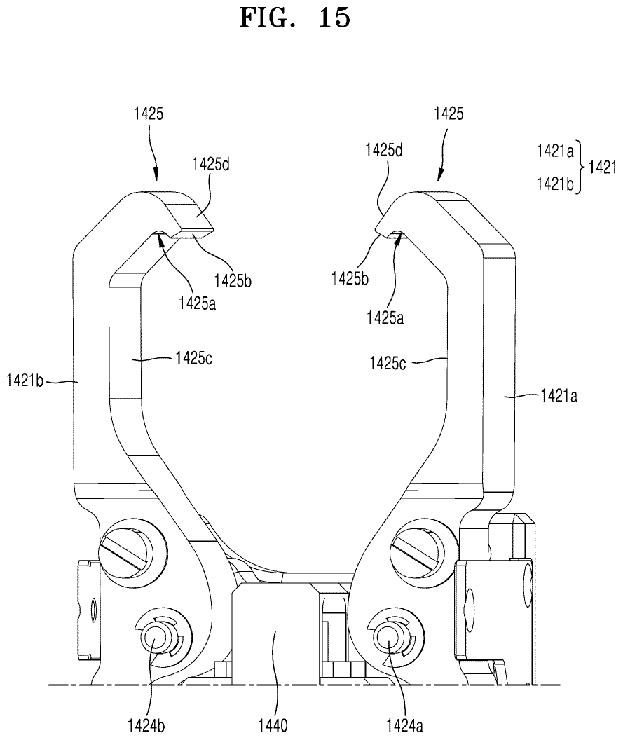

The groove 1522 may be formed in a height direction perpendicular to a circumferential direction of the mounting portion 1520 and may be formed to have a predefined width and depth. The groove 1522 may be engaged with edge portions (see 1425b of FIG. 15) of the first clamp 1420a and the second clamp 1420b to be described below.

In some embodiments, the mounting portion 1520 may include raised portions 1523 adjacent to the grooves 1522. The raised portions 1523 may be formed along the longitudinal direction of the grooves 1522. The raised portions 1523 may be engaged with recessed portions 1425a provided at end portions of the first clamp 1420a and the second clamp 1420b to be described below.

In some embodiments, the mounting portion 1520 may be formed as a single body, but may also be provided with a separable first body 1520a and a separable second body 1520b so that the first body 1520a and the second body 1520b are coupled to each other. For example, the pusher member contact part 1521 may be provided in the first body 1520a, and the grooves 1522 and the raised portions 1523 may be provided in the second body 1520b.

Hereinafter, the trocar holder 1400 according to an embodiment is described.

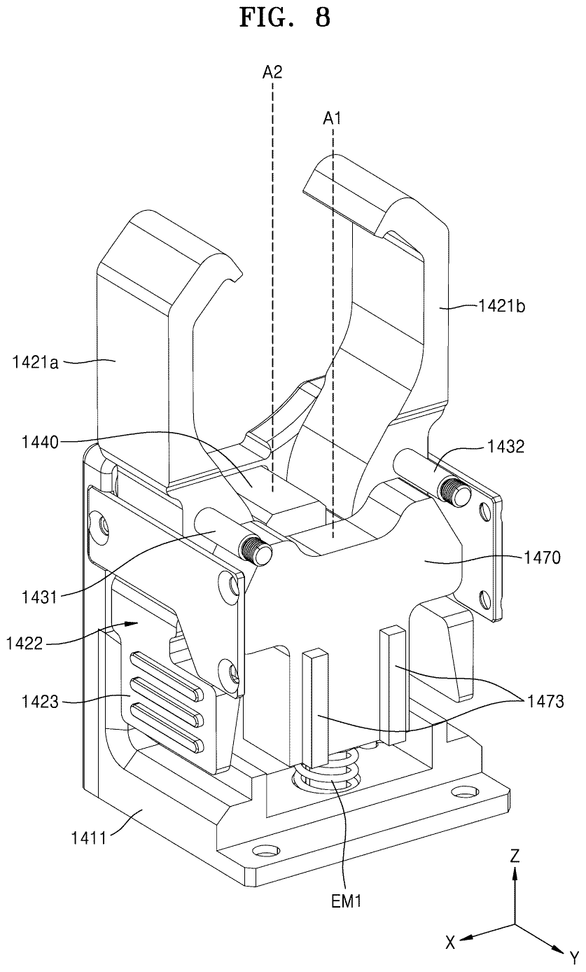

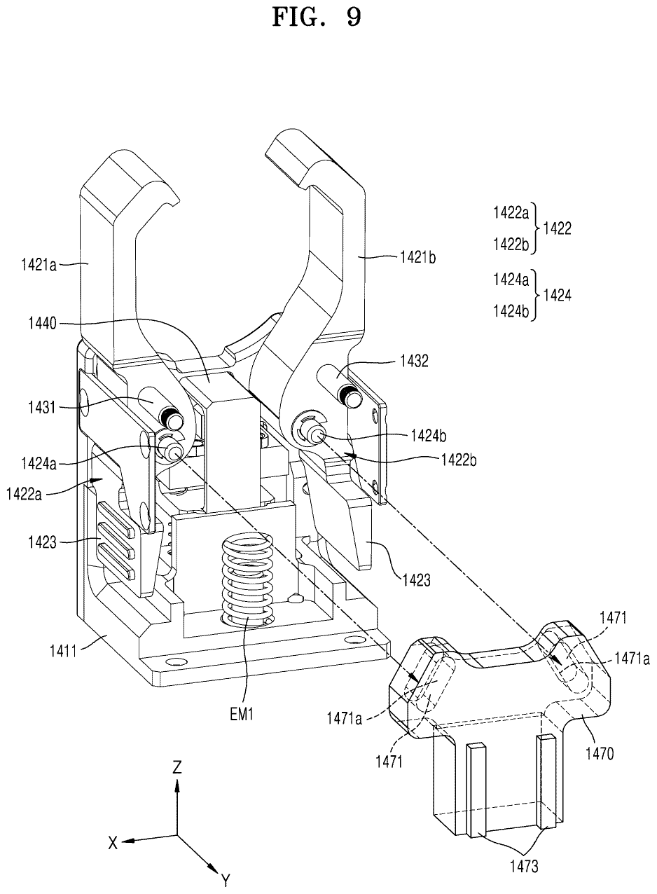

FIG. 7 is a perspective view illustrating the trocar holder 1400 according to an embodiment, and FIG. 8 is a perspective view illustrating a state in which an upper plate 1413 is removed from the trocar holder 1400 of FIG. 7. FIG. 9 is an exploded perspective view illustrating a sliding member 1470 separated from the trocar holder 1400 of FIG. 8.

Referring to FIGS. 7 to 9, the trocar holder 1400 according to an embodiment may include a clamp assembly CU, the sliding member 1470, and a holder body 1410.

The holder body 1410 may be coupled to an area of the arm assembly described above. For example, the holder body 1410 may be coupled to an area of a sliding arm.

The holder body 1410 may act as the body of the trocar holder 1400, and various components or devices constituting the trocar holder 1400 may be arranged in the holder body 1410.

In other words, the holder body 1410 may be a housing which accommodates various components or devices constituting the trocar holder 1400.

For example, the holder body 1410 may accommodate at least a portion of the clamp assembly CU to be described below. The sliding member 1470 and the pusher member 1440, which are to be described below, may be arranged inside the holder body 1410.

In describing the present disclosure, a portion close to the arm assembly is referred to as a proximal end, and a portion far from the arm assembly is referred to as a distal end. For example, a portion of the holder body 1410 close to the arm assembly is defined and described as a proximal end of the holder body 1410, and a portion of the holder body 1410 far from the arm assembly is defined and described as a distal end of the holder body 1410. Similarly, a portion of the clamp assembly CU close to the arm assembly is defined and described as a proximal end of the clamp assembly CU, and a portion of the clamp assembly CU far from the arm assembly is defined and described as a distal end of the clamp assembly CU.

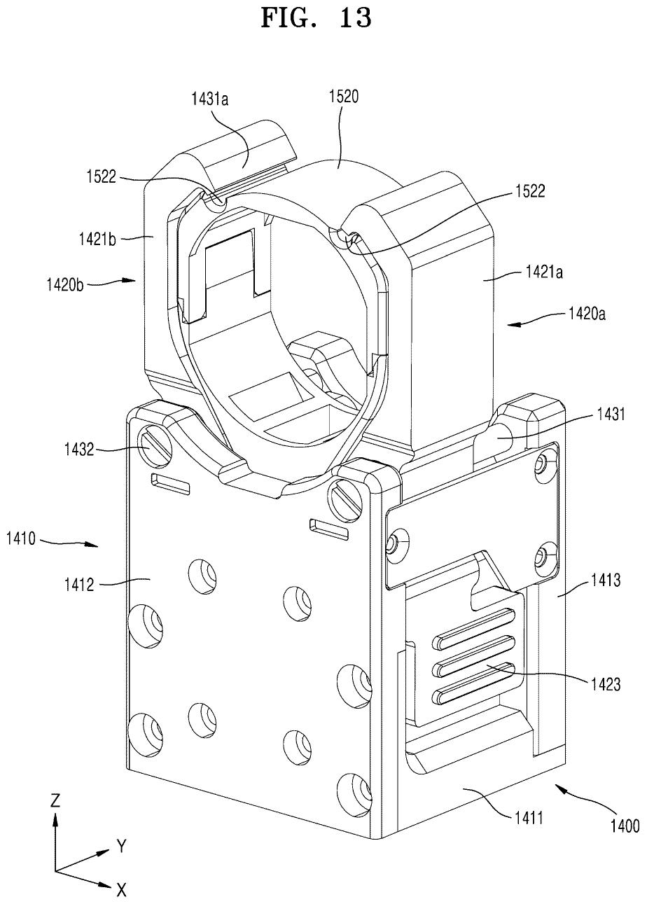

The holder body 1410 may include a body frame 1411, the upper plate 1413, and a lower plate (see 1412 of FIG. 13).

The main body frame 1411 may constitute a side wall on the proximal end side of the holder body 1410. The upper plate 1413 and the lower plate 1412 may be coupled to a side surface of the body frame 1411. In an embodiment, the lower plate 1412 or the upper plate 1413 may be formed integrally with the body frame 1411.

A first elastic member EM1 to be described below may be arranged in the body frame 1411. In other words, the body frame 1411, which is a coupling part to which the first elastic member EM1 is coupled, may support the first elastic member EM1.

The upper plate 1413 and the lower plate 1412 are parts to which rotation shafts to be described below are coupled, and the clamp assembly CU may be supported or fixed to the upper plate 1413 or the lower plate 1412.

The clamp assembly CU according to an embodiment may be a structure capable of gripping the trocar 1500 mounted on the trocar holder 1400. For example, the clamp assembly CU may be referred to as a grip unit.

The clamp assembly CU may include one or more clamps. However, in describing the present disclosure, a case where the clamp assembly CU includes two clamps is described as an example. For example, the clamp assembly CU may include a first clamp 1420a and a second clamp 1420b.

The clamp assembly CU according to an embodiment may be arranged at the distal end of the holder body 1410. For example, the first clamp 1420a and the second clamp 1420b may be at least partially accommodated in the holder body 1410.

The first clamp 1420a and the second clamp 1420b may be arranged opposite to each other and may be rotatable around different shafts. In other words, the first clamp 1420a and the second clamp 1420b may each be a rotating member. For example, the first clamp 1420a may be rotatable around a first rotation shaft 1431, and the second clamp 1420b may be rotatable around a second rotation shaft 1432.

The first rotation shaft 1431 and the second rotation shaft 1432 may be axially coupled to the holder body 1410. In other words, the rotation centers of the first clamp 1420a and the second clamp 1420b may be formed in an area of the holder body 1410.

For example, the first rotation shaft 1431 may be axially coupled to the vicinity of the center of the first clamp 1420a, the second rotation shaft 1432 may be axially coupled to the vicinity of the center of the second clamp 1420b, and the first rotation shaft 1431 and the second rotation shaft 1432 may be axially coupled to the holder body 1410.

The first rotation shaft 1431 and the second rotation shaft 1432 may be spaced apart from each other. The first rotation shaft 1431 and the second rotation shaft 1432 may be arranged parallel to each other.

The clamp assembly CU according to an embodiment may include a grip portion 1421 and an acting portion 1422. For example, the first clamp 1420a may include a first grip portion 1421a and a first acting portion 1422a, and the second clamp 1420b may include a second grip portion 1421b and a second acting portion 1422b.

The first grip portion 1421a may be a part which extends from the rotation center of the first clamp 1420a and grips and fixes the trocar 1500, and the first acting portion 1422a may be a part which extends to the opposite side of the first grip portion 1421a with respect to the rotation center of the first clamp 1420a.

Similarly, the second grip portion 1421b may be a part which extends from the rotation center of the second clamp 1420b and grips and fixes the trocar 1500, and the second acting portion 1422b may be a part which extends to the opposite side of the second grip portion 1421b with respect to the rotation center of the second clamp 1420b.

In the present disclosure, when common features of the first clamp 1420a and the second clamp 1420b are described, the first clamp 1420a and the second clamp 1420b are referred to as the clamp 1420. Similarly, the first grip portion 1421a and the second grip portion 1421b are referred to as the grip portion 1421, and the first acting portion 1422a and the second acting portion 1422b are referred to as the acting portion 1422.

The grip portion 1421 and the acting portion 1422 may be formed as a single member, and the grip portion 1421 and the acting portion 1422 may be distinguished based on the rotation center of the clamp 1420. In some embodiments, the grip portion 1421 may extend forward from the distal end side of the holder body 1410, and the acting portion 1422 may extend toward the proximal end side of the holder body 1410.

The clamp 1420 may be formed to be rotatable around the rotation shaft, and the acting portion 1422 may be a part which applies a force to the clamp 1420 so as to rotate the clamp 1420.

For example, a reaction member 1424 to be described below may be arranged in the acting portion 1422, and the clamp 1420 may be rotated by a force applied to the reaction member 1424.

In some embodiments, the acting portion 1422 may include a holding region 1423. The holding region 1423 may be provided so that the user holds and presses the holding region 1423. In other words, the holding region 1423 may be exposed to the outside of the holder body 1410 so as to be pressed from the outside. For example, the holding region 1423 may be arranged on the side surface of the holder body 1410. The user may spread the grip portion 1421 left and right by pressing the holding region 1423 arranged on the side surface of the holder body 1410. Details thereof are described below.

In some embodiments, the clamp assembly CU according to an embodiment may further include the reaction member 1424.

The reaction member 1424 may be a member which comes into contact with the sliding member 1470 to be described below and exchanges a force with the sliding member 1470. In other words, the reaction member 1424 and the sliding member 1470 may be movable by the force acting between the reaction member 1424 and the sliding member 1470.

In an embodiment, the reaction member 1424 may be arranged in each of the first clamp 1420a and the second clamp 1420b and may come into contact with the sliding member 1470 to be described below.

For example, a pair of reaction members 1424 may be respectively arranged in the first acting portion 1422a and the second acting portion 1422b. For example, a first reaction member 1424a may be arranged in the first acting portion 1422a and spaced apart from the first rotation shaft 1431, and a second reaction member 1424b may be arranged in the second acting portion 1422b and spaced apart from the second rotation shaft 1432. The first reaction member 1424a and the second reaction member 1424b may be arranged closer to the rotation shaft than the holding region 1423, but the concept of the present disclosure is not limited thereto.

The reaction member 1424 may be formed in a cylindrical shape so that the side surface of the reaction member 1424 may come into contact with the sliding member 1470. The reaction member 1424 may be arranged parallel to the rotation shaft. In other words, the first reaction member 1424a may be arranged parallel to the first rotation shaft 1431, and the second reaction member 1424b may be arranged parallel to the second rotation shaft 1432.

The first reaction member 1424a may be inserted through the first clamp 1420a, and the second reaction member 1424b may be inserted through the second clamp 1420b. From another viewpoint, the reaction member 1424 may be arranged parallel to the rotation shaft and inserted through the clamp 1420. However, unlike the rotation shaft, the reaction member 1424 is not fixedly coupled to the holder body 1410 and may be relatively movable with respect to the holder body 1410.

For example, an end portion of the reaction member 1424 may come into contact with the sliding member 1470, and another end portion of the reaction member 1424 may come into contact with the lower plate 1412. In an embodiment, the lower plate 1412 may form a space in which the other end portion of the reaction member 1424 may be inserted and moved. Although not illustrated, a moving coupling hole where the end portion of the shaft is inserted and moved may be formed. The moving coupling hole may guide a path along which the reaction member 1424 is moved.

The sliding member 1470 may be arranged in the holder body 1410 and connected to the first clamp 1420a and the second clamp 1420b. For example, the sliding member 1470 may be connected to the first reaction member 1424a and the second reaction member 1424b.

The sliding member 1470 may be moved between the proximal end and the distal end along a first axis A1. In other words, the sliding member 1470 may be a member which performs a linear motion.

The sliding member 1470 may cause the rotation motions of the first clamp 1420a and the second clamp 1420b as the sliding member 1470 is moved along the first axis A1.

For example, the sliding member 1470 may allow the first clamp 1420a and the second clamp 1420b to be rotated opposite to each other in a state of being symmetrically arranged.

From another viewpoint, the sliding member 1470 may rotate the first clamp 1420a and the second clamp 1420b while the two clamps maintain the symmetrical state. In other words, the first clamp 1420a and the second clamp 1420b may be rotated while maintaining the symmetrical state by the sliding member 1470.

The first grip portion 1421a and the second grip portion 1421b may be moved closer to or farther away from each other by the motion of the sliding member 1470. Details thereof are described below.

The sliding member 1470 may include a pair of first inclined portions 1471a formed symmetrically with respect to the first axis A1 at the front end.

The pair of first inclined portions 1471a may be inclined so as to gradually approach the first axis A1 along the front. In other words, the first inclined portion 1471a may be closer to the first axis A1 on the proximal end side than on the distal end side.

From another viewpoint, the distance between the pair of first inclined portions 1471a may increase from the distal end side to the proximal end side.

The sliding member 1470 may come into contact with the reaction member 1424 and may be pressed by the reaction member 1424 or may press the reaction member 1424. For example, the first inclined portion 1471a may be pressed by the reaction member 1424 or may press the reaction member 1424.

In an embodiment, the sliding member 1470 may include a pair of guide holes 1471 formed symmetrically with respect to the first axis A1 at the front end. At least one surface of the guide hole 1471 may include the first inclined portion 1471a described above. In other words, the sliding member 1470 may include a pair of guide holes 1471 having at least one surface including the first inclined portion 1471a. From another viewpoint, the first inclined portion 1471a may form at least one surface of the guide hole 1471.

For example, the guide hole 1471 may be formed on a surface of the sliding member 1470 opposite the acting portion 1422. The guide hole 1471 may be formed to have a predefined depth and a length corresponding to the first inclined portion 1471a.

At least an area of the reaction member 1424 may be inserted into the guide hole 1471 so that the reaction member 1424 is movable within the guide hole 1471.

For example, an end portion of the reaction member 1424 may be inserted into the guide hole 1471 so that the reaction member 1424 is movable along the longitudinal direction of the guide hole 1471. For example, the guide hole 1471 may guide the movement of the reaction member 1424 and may restrict the range of movement of the reaction member 1424.

In some embodiments, the sliding member 1470 may further include a protrusion 1473 which guides the linear motion of the sliding member 1470.

For example, the sliding member 1470 may include the protrusion 1473 on the opposite side of the surface where the guide hole 1471 is formed. In other embodiments, the protrusion 1473 may be provided on the side surface of the sliding member 1470. The protrusion 1473 may be formed in a rectangular shape parallel to the first axis A1. However, the concept of the present disclosure is not limited thereto, and the protrusion 1473 may be formed in a cylindrical shape or other shapes which allow the protrusion 1473 to be movable while being inserted into a sliding groove 1413a to be described below.

In some embodiments, the upper plate 1413 may have a sliding groove 1413a in the inner surface opposite the sliding member 1470. The sliding groove 1413a may be formed in a direction parallel to the first axis A1.

The protrusion 1473 of the sliding member 1470 may perform a linear motion in a state of being engaged with the sliding groove 1413a of the upper plate 1413. For example, the sliding groove 1413a may guide the sliding member 1470 so that the sliding member 1470 performs a linear motion. Therefore, the sliding member 1470 may be moved in a straight direction along the first axis A1.

In some embodiments, the holder body 1410 may further include a first elastic member EM1 which presses the sliding member 1470 toward the distal end side. In other words, the first elastic member EM1 may provide an elastic force to cause the sliding member 1470 to move forward.

An end portion of the first elastic member EM1 may be coupled to the body frame 1411, and another end portion of the first elastic member EM1 may be coupled to the sliding member 1470.

Hereinafter, the force acting on the sliding member 1470 and the clamp 1420 of the trocar holder 1400 according to an embodiment and the operating process thereof are described.

The force acting on the clamp 1420 may be generated by the force transmitted from the first elastic member EM1 to the sliding member 1470. For example, the elastic force of the first elastic member EM1 may be transmitted to the clamp 1420 through the sliding member 1470. The trocar 1500 may be fixed to the trocar holder 1400 by the force of the clamp 1420.

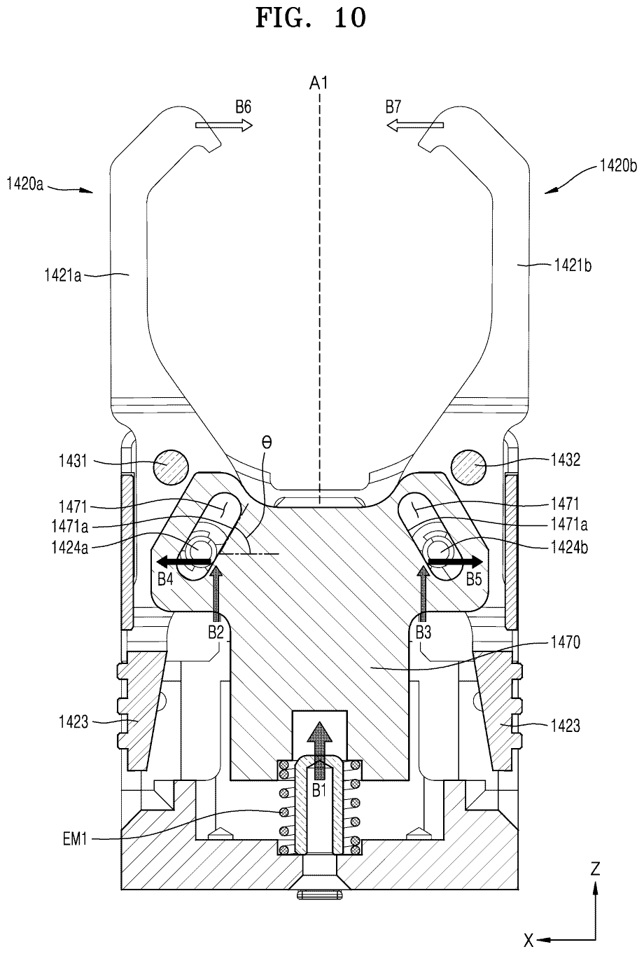

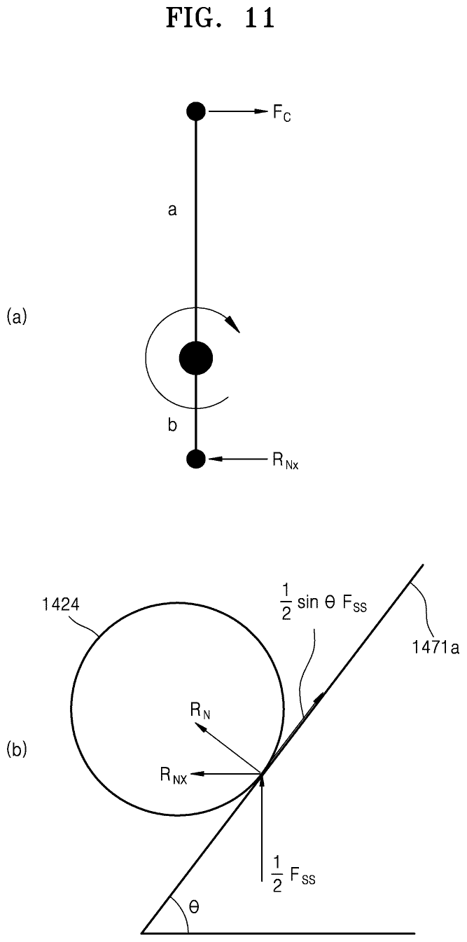

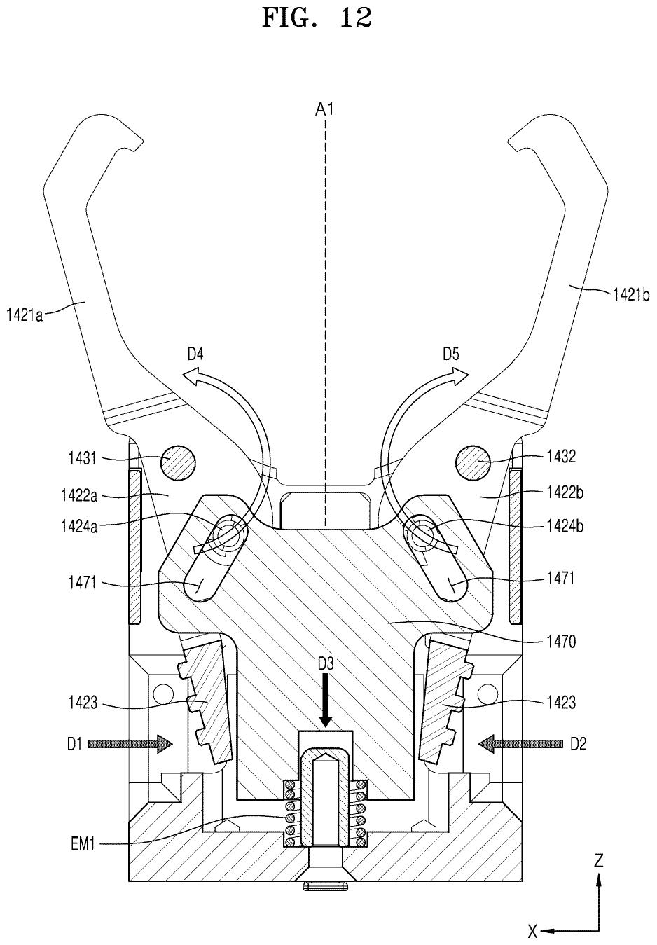

FIG. 10 is a cross-sectional view illustrating a cross-section of the trocar holder 1400 of FIG. 8. FIG. 11 is a diagram illustrating the force acting between the sliding member 1470 and the clamp 1420 of the trocar holder 1400 according to an embodiment. FIG. 12 is a cross-sectional view illustrating an opening operation of the trocar holder 1400 of FIG. 10.

Referring to FIG. 10, the first elastic member EM1 may provide an elastic force to the sliding member 1470 in a direction of an arrow B1. The sliding member 1470 may apply a force to the reaction member 1424 through the first inclined portion 1471a. In other words, when the sliding member 1470 is moved forward toward the distal end side, the first inclined portion 1471a may apply a force to the reaction member 1424.

From another viewpoint, the reaction member 1424 may come into contact with the first inclined portion 1471a and may be pressed by the first inclined portion 1471a. For example, the first inclined portion 1471a on the left side may transmit a force caused by sliding to the first reaction member 1424a in a direction of an arrow B2, and the first inclined portion 1471a on the right side may transmit a force caused by sliding to the second reaction member 1424b in a direction of an arrow B3.

A horizontal force may act on the first reaction member 1424a in a direction of an arrow B4, and a force in a direction of an arrow B6 may be generated at an end action point of the first clamp 1420a by the force acting on the first reaction member 1424a.

Similarly, a horizontal force may act on the second reaction member 1424b in a direction of an arrow B5, and a force in a direction of an arrow B7 may be generated at an end action point of the second clamp 1420b by the force acting on the second reaction member 1424b.

In other words, the horizontal force of the first reaction member 1424a may rotate the first clamp 1420a, and the horizontal force of the second reaction member 1424b may rotate the second clamp 1420b.

As a result, when the sliding member 1470 is moved forward toward the distal end side, the reaction member 1424 may be spaced apart from the first axis A1.

The rotation of the first grip portion 1421a and the second grip portion 1421b in a direction toward each other is defined and described as internal rotation, and the rotation of the first grip portion 1421a and the second grip portion 1421b in a direction away from each other is defined and described as external rotation.

For example, in the trocar holder 1400 illustrated in FIG. 10, clockwise rotation of the first clamp 1420a is described as internal rotation, and counterclockwise rotation of the first clamp 1420a is described as external rotation.

The trocar holder 1400 according to an embodiment may receive a force to move the sliding member 1470 toward the distal end side by the first elastic member EM1, and accordingly, a force in the direction of internal rotation may act on the first clamp 1420a and the second clamp 1420b.

FIG. 11 illustrates a free body diagram of the force generated in the clamp 1420. Referring to FIG. 11(a), the length from the rotation center to the end action point of the clamp 1420 may be represented by a, and the length from the rotation center to the action point of the reaction member 1424 may be represented by b. At this time, the force (F_{C}) at the end action point of the clamp 1420 and the horizontal force (R_{NX}) acting on the reaction member 1424 satisfy a relationship aF_{C}=bR_{NX} according to the proportion of the vertical length from the rotation center of the clamp 1420.

FIG. 11(b) illustrates the force acting on the reaction member 1424 when the first inclined portion 1471a and the reaction member 1424 in FIG. 10 come into contact with each other.

Referring to FIGS. 10 to 11(b), the angle between the horizontal surface perpendicular to the first axis A1 and the first inclined portion 1471a may be represented by θ. When the elastic force in the direction of the arrow B1 provided by the first elastic member EM1 to the sliding member 1470 is represented by F_{SS}, the force caused by sliding in the direction of the arrow B2 is represented by ½ F_{SS}.

When the first inclined portion 1471a of the sliding member 1470 and the reaction member 1424 come into contact with each other, the sliding reaction force (R_{N}) may be generated through the sliding member 1470, and the horizontal force (R_{NX}) of R_{N} may rotate the clamp 1420.

R_{N} may be represented by

$${R}_{N} = \frac{1}{2} cos θ {F}_{SS} ,$$

and R_{NX} may be represented by R_{NX}=R_{N} sin θ.

As a result, F_{C} is organized into F_{SS} as follows.

$${F}_{c} = \frac{b}{a} {R}_{NX} = \frac{1}{2} \cdot \frac{b}{a} sin θcos θ {F}_{SS}$$

As described above, the elastic force of the first elastic member EM1 transmitted through the sliding member 1470 may be converted into a force which causes the first grip portion 1421a and the second grip portion 1421b to grip the trocar 1500.

In contrast, referring to FIG. 12, when the first clamp 1420a and the second clamp 1420b are externally rotated, the sliding member 1470 may be moved toward the proximal end side.

For example, when the first clamp 1420a is externally rotated in the direction of the arrow D4 and the second clamp 1420b is externally rotated in the direction of the arrow D5, the first reaction member 1424a and the second reaction member 1424b may press the first inclined portion 1471a. For example, when the first grip portion 1421a and the second grip portion 1421b are spaced apart from each other, the first acting portion 1422a and the second acting portion 1422b may come close to each other, and the first reaction member 1424a and the second reaction member 1424b may be moved closer to the first axis A1. When the reaction member 1424 is moved closer to the first axis A1, the reaction member 1424 may apply a force to the first inclined portion 1471a. In this case, the sliding member 1470 may be moved backward toward the proximal end side along a direction of an arrow D3, and the first elastic member EM1 may be compressed.

When the first clamp 1420a and the second clamp 1420b are externally rotated, there may be a case where a force acts on the first grip portion 1421a and the second grip portion 1421b and a case where a force acts on the first acting portion 1422a and the second acting portion 1422b.

As described below, when the trocar 1500 is mounted on the trocar holder 1400, the first grip portion 1421a and the second grip portion 1421b may be spaced apart from each other. In other words, when the first grip portion 1421a and the second grip portion 1421b come close to each other and the trocar 1500 is pushed between the first grip portion 1421a and the second grip portion 1421b, the first grip portion 1421a and the second grip portion 1421b may be spread from each other.

In this case, as described above, a force may act in a direction in which the first grip portion 1421a and the second grip portion 1421b are spaced apart from each other, and the first reaction member 1424a and the second reaction member 1424b may apply a force to the sliding member 1470 so that the sliding member 1470 may be moved backward.

In some embodiments, the user may unmount the trocar 1500 from the trocar holder 1400 by pressing the holding region 1423. When the user presses the holding region 1423, a force may act to bring the first acting portion 1422a and the second acting portion 1422b closer to each other. In other words, a force may act in a direction in which the first reaction member 1424a of the first acting portion 1422a and the second reaction member 1424b of the second acting portion 1422b come close to the first axis A1. Therefore, the sliding member 1470 may be moved backward toward the proximal end side by the reaction member 1424.

When the first elastic member EM1 is compressed, the sliding member 1470 may receive a force to move forward toward the distal end side due to the restoring force of the first elastic member EM1. Therefore, when the force of the reaction member 1424 pressing the first inclined portion 1471a is removed or weakened, the sliding member 1470 may be moved forward. Finally, the first acting portion 1422a and the second acting portion 1422b may be forced away from the first axis A1 by the sliding member 1470, and thus, the first clamp 1420a and the second clamp 1420b may be externally rotated.

Hereinafter, the pusher member 1440 and the clamp assembly CU of the trocar holder 1400 according to an embodiment are described.

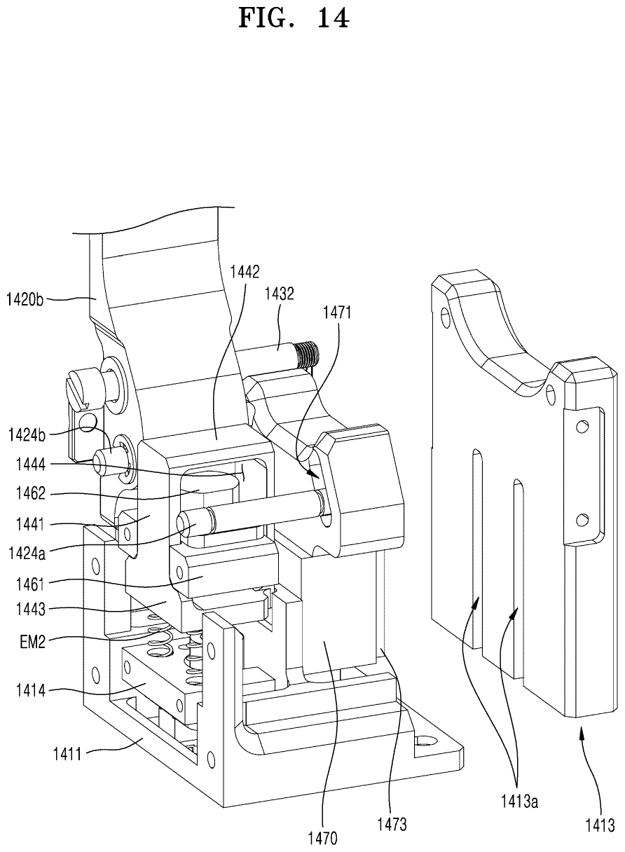

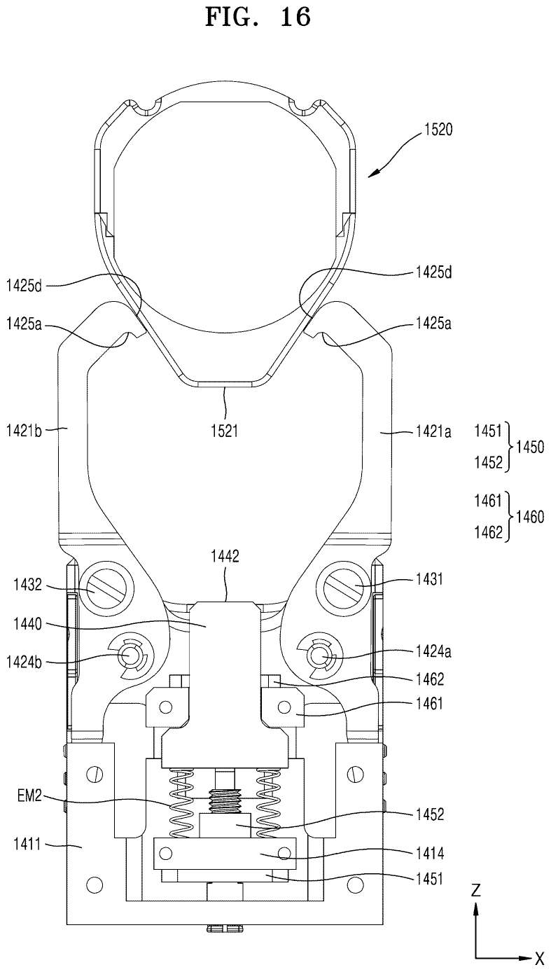

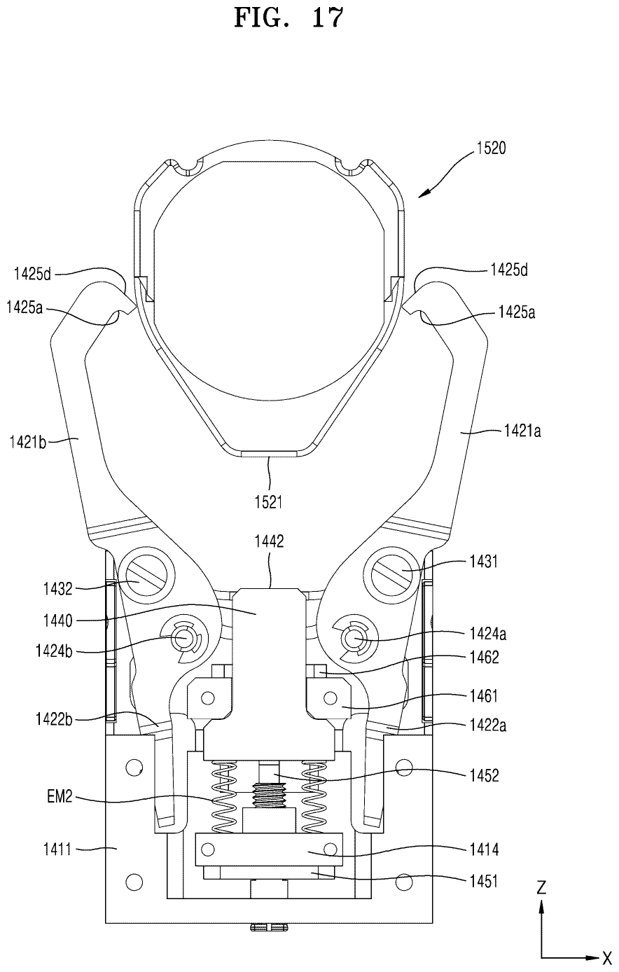

FIG. 13 is a perspective view illustrating the trocar holder 1400 and the mounting portion 1520 of the trocar 1500 of FIG. 5, FIG. 14 is an exploded perspective view illustrating a partial configuration including the pusher member 1440 of the trocar holder 1400 of FIG. 13, FIG. 15 is an enlarged perspective view illustrating the clamp 1420 of the trocar holder 1400 of FIG. 13. FIGS. 16 to 18 are diagrams illustrating a state of the trocar holder 1400 when the trocar 1500 is mounted on the trocar holder 1400, according to an embodiment.

In FIGS. 13 and 16 to 18, the configuration of the trocar 1500 except for the mounting portion 1520 of the trocar 1500 is omitted.

Referring to FIGS. 13 and 14, the trocar holder 1400 according to an embodiment may further include a pusher member 1440 and a second elastic member EM2.

The pusher member 1440 may be arranged inside the holder body 1410. For example, the pusher member 1440 may be arranged between the first clamp 1420a and the second clamp 1420b. For example, the pusher member 1440 may be arranged between the first acting portion 1422a and the second acting portion 1422b. From another viewpoint, the pusher member 1440 may be arranged between the lower plate 1412 and the sliding member 1470.

The pusher member 1440 may be moved along a second axis A2 parallel to the first axis A1. For example, the pusher member 1440 may be moved between the proximal end side and the distal end side of the holder body 1410.

In some embodiments, an end portion of the second elastic member EM2 may be fixed to the holder body 1410, and another end portion of the second elastic member EM2 may be coupled to the pusher member 1440. For example, a second elastic member support part 1414 may be provided in the body frame 1411, and the other end portion of the second elastic member EM2 may be coupled to the second elastic member support part 1414.

The second elastic member EM2 may press the pusher member 1440 toward the distal end side. Therefore, the pusher member 1440 may be moved toward the distal end side until the pusher member 1440 interferes with a second detector holder 1461. From another viewpoint, the second detector holder 1461 may restrict the pusher member 1440 from moving further toward the distal end side. For example, a protrusion 1443 of the pusher member 1440 may interfere with the second detector holder 1461.

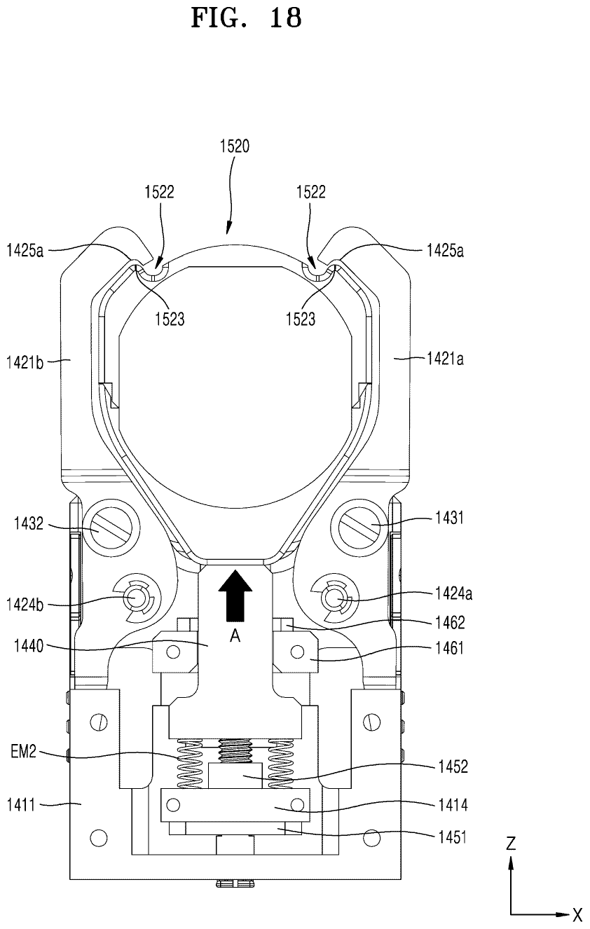

When the trocar 1500 is mounted on the trocar holder 1400, the pusher member 1440 may come into contact with the mounting portion 1520 of the trocar 1500. For example, a contact surface 1442 of the pusher member 1440 may come into contact with the pusher member contact part 1521 of the mounting portion 1520 described above. Referring to FIG. 18, when the mounting portion 1520 of the trocar 1500 pushes the pusher member 1440 so that the pusher member 1440 is moved toward the proximal end side, the second elastic member EM2 may be compressed. In contrast, due to the elastic force of the second elastic member EM2, the pusher member 1440 may apply a force to push the mounting portion 1520 toward the distal end side. For example, the pusher member 1440 may press the trocar 1500 toward the distal end side.

As described above, the trocar 1500 may try to move back toward the distal end side due to the force of the pusher member 1440 pressing the trocar 1500 toward the distal end side, but the movement of the trocar 1500 may be restricted by the first grip portion 1421a and the second grip portion 1421b and the trocar 1500 may be brought into close contact with the first grip portion 1421a and the second grip portion 1421b.

Referring to FIG. 15, the first clamp 1420a and the second clamp 1420b according to an embodiment may respectively include fastening portions 1425 at end portions on the distal end side. The fastening portion 1425 may be formed to be curved inward. For example, the grip portion 1421 may have a shape to extend from the rotation center to the distal end side and to bend toward the opposite grip portion 1421 at the end portion on the distal end side or to extend to the proximal end side. For example, the fastening portion 1425 may be formed in a hook shape.

The fastening portion 1425 may include a recessed portion 1425a and an edge 1425b. As illustrated, the recessed portion 1425a, which is a portion recessed concavely toward the distal end side of the grip portion 1421, may be engaged with the raised portion 1523 of the mounting portion 1520 described above.

The edge 1425b, which is the end portion of the grip portion 1421, may be engaged with the groove 1522 of the mounting portion 1520 described above. The edge 1425b may be directed toward the opposite other grip portion 1421 or toward the proximal end side.

The first clamp 1420a and the second clamp 1420b may respectively include inner surfaces 1425c corresponding to the shape of the mounting portion 1520 of the trocar 1500. For example, the inner surface 1425c of the first grip portion 1421a and the inner surface 1425c of the second grip portion 1421b may be formed in a shape corresponding to the side surface of the mounting portion 1520 so as to surround the side surface of the mounting portion 1520.

In some embodiments, the clamp assembly CU may include a second inclined portion 1425d which comes into contact with the mounting portion 1520 of the trocar 1500. For example, the second inclined portion 1425d bent toward the space between the first clamp 1420a and the second clamp 1420b may be formed at the end portion of each of the first clamp 1420a and the second clamp 1420b on the distal end side. For example, the first clamp 1420a and the second clamp 1420b may form the second inclined portion 1425d which is gradually inclined away from the second axis A2 along the end portion on the distal end side. The second inclined portion 1425d may form a surface of the fastening portion 1425.

In some embodiments, the second inclined portions 1425d may be arranged at the same distance with respect to the second axis A2. In other words, a pair of second inclined portions 1425d may be provided opposite to each other.

Referring to FIGS. 16 to 18, when the trocar 1500 is mounted on the trocar holder 1400, the side surface of the mounting portion 1520 may come into contact with the second inclined portion 1425d. Because the mounting portion 1520 on which the pusher member contact part 1521 is formed protrudes in a shape which gradually narrows, the mounting portion 1520 around the pusher member contact part 1521 may not come into contact with the second inclined portion 1425d. In some embodiments, the mounting portion 1520 may be formed to have a width which gradually increases to correspond to the pair of second inclined portions 1425d. Therefore, when the mounting portion 1520 is inserted between the first grip portion 1421a and the second grip portion 1421b, the mounting portion 1520 may be slidingly inserted along the second inclined portion 1425d while coming into contact with the second inclined portion 1425d.

Therefore, the mounting portion 1520 may be easily inserted between the first grip portion 1421a and the second grip portion 1421b. At this time, when the mounting portion 1520 is pushed between the first grip portion 1421a and the second grip portion 1421b, the first grip portion 1421a and the second grip portion 1421b may be spread from each other.

Therefore, according to an embodiment, the user may fasten the trocar 1500 to the trocar holder 1400 by simply pushing the trocar 1500 into the trocar holder 1400.

When the trocar 1500 is fully inserted between the first grip portion 1421a and the second grip portion 1421b, the first grip portion 1421a and the second grip portion 1421b may be internally rotated to grip the trocar 1500 by the first elastic member EM1 and the sliding member 1470, as described above. In some embodiments, the first grip portion 1421a and the second grip portion 1421b may maintain the fixed state of the trocar 1500 by continuously pressing the mounting portion 1520 from opposite sides of the mounting portion 1520.

Because the mounting portion 1520 is pushed toward the distal end side by the pusher member 1440, the raised portion 1523 of the mounting portion 1520 may come into close contact with the recessed portion 1425a of the grip portion 1421, and the groove 1522 of the mounting portion 1520 may come into close contact with the edge 1425b of the grip portion 1421. For example, the trocar 1500 may come into contact with the fastening portion 1425, and the fastening portion 1425 may restrict the movement of the trocar 1500 toward the distal end side.

In other words, because the mounting portion 1520 interferes with the fastening portion 1425 of the grip portion 1421 in a state of being arranged between the grip portions 1421, and thus, is not pushed further toward the distal end side, the mounting portion 1520 may be further pressed by the pusher member 1440 between the fastening portion 1425 and the pusher member 1440. Accordingly, the trocar holder 1400 according to an embodiment may further increase the fastening force of the trocar 1500 when the trocar 1500 is fastened to the trocar holder 1400.

Because the trocar holder 1400 according to an embodiment fastens the trocar 1500 by pushing the trocar 1500 between the grip portions 1421, no additional manipulation is required, thereby improving convenience in use.

In some embodiments, the trocar holder 1400 according to an embodiment may facilitate mounting of the trocar 1500 and increase the fastening force for maintaining the fixed state after mounting of the trocar 1500, thereby preventing detachment of the trocar 1500.

According to an embodiment, the user may release the grip of the trocar 1500 by pressing the holding region 1423 of the clamp 1420. Therefore, the trocar holder 1400 according to an embodiment may allow the trocar 1500 to be easily removed from the trocar holder 1400, thereby improving convenience of mounting and unmounting.

In some embodiments, the trocar holder 1400 according to an embodiment may include at least one of a first detector 1450 or a second detector 1460, which detects whether the trocar 1500 is mounted.

The first detector 1450 may be arranged inside the holder body 1410. For example, the first detector 1450 may be arranged in the body frame 1411. The first detector 1450 may be arranged in an area of the second elastic member support part 1414.

The first detector 1450 may be a detector which is operated by physical contact and pressure. The first detector 1450 may be operated when the pusher member 1440 is pressed and moved backward toward the proximal end side. For example, as illustrated in FIG. 18, when the mounting portion 1520 of the trocar 1500 presses the pusher member 1440 so that the pusher member 1440 is moved backward toward the proximal end side, the pusher member 1440 may press the switch 1452. The detector 1451 may be operated to detect the pressed switch 1452. The switch 1452 may be, for example, a pogo pin-type switch, but the present disclosure is not necessarily limited thereto.

The second detector 1460 may detect a target object in a non-contact manner. For example, the second detector 1460 may detect whether the trocar 1500 is mounted or unmounted. For example, the second detector 1460 may include a short-range communication module 1462. For example, the short-range communication module 1462 may use near field communication (NFC) technology. For example, an NFC tag may be embedded in the trocar 1500 and an NFC antenna may be arranged in the short-range communication module 1462, so that the trocar 1500 may be detected when the trocar 1500 is mounted on the trocar holder 1400.

The second detector 1460 may be arranged in the second detector holder 1461. The second detector holder 1461 may be coupled and fixed to the holder body 1410. For example, the second detector holder 1461 may be coupled to the lower plate 1412.

In some embodiments, the pusher member 1440 may have a shape in which a portion of the body is penetrated. In other words, the pusher member 1440 may include a cavity 1444. The second detector holder 1461 may be arranged to pass through the cavity 1444 of the pusher member 1440. For example, the second detector holder 1461 may have an area arranged inside the cavity 1444 of the pusher member 1440 and an area exposed to the outside of the pusher member 1440.

When the pusher member 1440 is moved along the second axis A2, the second detector 1460 may be arranged not to interfere with the pusher member 1440. However, a portion of the second detector holder 1461 may interfere with the lower protrusion 1443 of the pusher member 1440 and restrict the movement of the pusher member 1440.

The trocar holder 1400 according to an embodiment may include both the first detector 1450 and the second detector 1460 so as to detect whether the trocar 1500 is mounted. Furthermore, the trocar holder 1400 may also provide information about the type of the mounted trocar 1500 by reading information about the mounted trocar 1500 through the second detector 1460.

According to the present disclosure, the trocar holder for the surgical robot, which facilitates mounting and unmounting of the trocar and improves the fastening force of the trocar, may be provided.

The present disclosure has been described with reference to some embodiments. Those of ordinary skill in the art will understand that the present disclosure may be implemented in modified forms without departing from the essential features of the present disclosure. Therefore, the disclosed embodiments should be considered in an illustrative sense rather than a restrictive sense. The scope of the present disclosure is indicated in the claims rather than the foregoing description, and all differences within the scope equivalent thereto should be construed as falling within the present disclosure.

What is claimed is:

1. A trocar holder for a surgical robot, the trocar holder comprising:
a clamp assembly comprising a first clamp and a second clamp arranged to face each other and configured to be rotatable around different shafts;
a sliding member connected to the first clamp and the second clamp, and configured to move between a proximal end and a distal end along a first axis to cause rotational motions of the first clamp and the second clamp; and
a holder body in which the sliding member and at least a portion of the clamp assembly are disposed.

2. The trocar holder of claim 1,
wherein the clamp assembly further comprises reaction members respectively disposed on the first clamp and the second clamp and configured to contact the sliding member, and
wherein the reaction members and the sliding member are configured to move relative to each other by a force acting between the reaction members and the sliding member.

3. The trocar holder of claim 2,
wherein the sliding member comprises a pair of first inclined portions formed symmetrically with respect to the first axis at a first end, and
wherein each of the reaction members is configured to contact a respective one of the pair of first inclined portions, and to be pressed by the respective one of the pair of first inclined portions or to press the respective one of the pair of first inclined portions.

4. The trocar holder of claim 3,
wherein, when the sliding member moves in a direction toward the distal end, the pair of first inclined portions press the reaction members, and
wherein, when the reaction members move closer to the first axis, the reaction members press the pair of first inclined portions.

5. The trocar holder of claim 4, wherein, when the sliding member moves in the direction toward the distal end, the reaction members move in a direction away from the first axis, and the first clamp and the second clamp are internally rotated.

6. The trocar holder of claim 4, wherein, when the first clamp and the second clamp are externally rotated, the reaction members move in a direction closer to the first axis, and the sliding member moves in a direction toward the proximal end.

7. The trocar holder of claim 3,
wherein the sliding member comprises a pair of guide holes having at least one surface including the respective one of the pair of first inclined portions, and
wherein at least one portion of each of the reaction members is inserted into a respective one of the pair of guide holes and is movable therein.

8. The trocar holder of claim 3, wherein a distance between the pair of first inclined portions increases from a distal end side toward a proximal end side.

9. The trocar holder of claim 2, wherein each of the reaction members has a cylindrical shape, and a side surface of each of the reaction members contacts the sliding member.

10. The trocar holder of claim 2,
wherein the first clamp includes:
a first grip portion extending from a rotation center of the first clamp; and
a first acting portion extending to an opposite side of the first grip portion with respect to the rotation center of the first clamp, and
wherein the second clamp includes:
a second grip portion extending from a rotation center of the second clamp; and
a second acting portion extending to an opposite side of the second grip portion with respect to the rotation center of the second clamp.

11. The trocar holder of claim 10, wherein the reaction members are respectively disposed on each of the first acting portion and the second acting portion.

12. The trocar holder of claim 10,
wherein each of the first acting portion and the second acting portion provides a holding region configured to be pressed from an outside, and
wherein, when the holding region is pressed, the first grip portion and the second grip portion are rotated in a direction away from each other.

13. The trocar holder of claim 1, wherein the first clamp and the second clamp are rotated by the sliding member while maintaining a symmetrical state.

14. The trocar holder of claim 1, wherein the holder body comprises a first elastic member configured to press the sliding member in a direction toward the distal end.

15. The trocar holder of claim 1, wherein the holder body further comprises:
a pusher member configured to move along a second axis parallel to the first axis; and
a second elastic member configured to press the pusher member in a direction toward the distal end,
wherein the pusher member is configured to contact a mounting portion of a trocar when the trocar is mounted on the trocar holder.

16. The trocar holder of claim 15, wherein the pusher member is configured to press the mounting portion in the direction toward the distal end.

17. The trocar holder of claim 16, wherein each of the first clamp and the second clamp includes a fastening portion formed to be curved inwardly at a side toward the distal end,
wherein the fastening portion configured to contact the trocar and restrict movement of the trocar in the direction toward the distal end.

18. The trocar holder of claim 15,
wherein the holder body further comprises a first detector configured to detect whether the trocar is mounted, and
wherein the first detector is operated when the pusher member moves in a direction toward the proximal end.

19. The trocar holder of claim 1, wherein a second inclined portion bent toward a space between the first clamp and the second clamp is formed at an end portion of each of the first clamp and the second clamp toward the distal end.

20. The trocar holder of claim 1, wherein each of the first clamp and the second clamp includes an inner surface corresponding to a shape of a mounting portion of a trocar.
